(19) United States
(12) Patent Application Publication
(10) Pub. No.: US 2024/0316060 A1
(43) Pub. Date: Sep. 26, 2024

(54) LASER AND BCL-2 INHIBITORS COMBINATION TO TREAT SOLID TUMORS

(71) Applicant: Board of Regents, The University of Texas System, Austin, TX (US)

(72) Inventors: Md Nurunnabi, El Paso, TX (US); Himanshu Bhatt, El Paso, TX (US)

(21) Appl. No.: 18/612,909

(22) Filed: Mar. 21, 2024

Related U.S. Application Data
(60) Provisional application No. 63/491,687, filed on Mar. 22, 2023.

(51) Int. Cl. A61K 31/5377; A61K 9/70

(52) U.S. Cl. CPC A61K 31/5377; A61K 9/7007

(57) ABSTRACT

Treating solid tumors includes administering to a mammal in need of treatment a compound including at least one BCL-2/BCL-xl inhibitor chemically bonded on a 2D nanosheet. The compound can include Navitoclax deposited on 2D nanosheets of boron nitride to form a nanosphere via π-π stacking.

### CROSS-REFERENCE TO RELATED APPLICATION

Referring to the application data sheet filed herewith, this application claims a benefit of priority under 35 U.S.C. 119(e) from co-pending provisional patent application U.S. Ser. No. 63/491,687, filed Mar. 22, 2023, the entire contents of which are hereby expressly incorporated herein by reference for all purposes.

### BACKGROUND INFORMATION

### 1. Field

The present invention relates generally to the field of medicine and disease treatment. More particularly, it concerns methods and compositions for treating solid tumors.

### 2. Background

The major challenge of treating solid tumors of any kind, is to deliver the anticancer therapeutics to the deeper layers of the solid tumor. Interstitial pressure severely limits the ability of anticancer therapeutics to penetrate solid tumors. The interstitial pressure can even repulse the anticancer therapeutics.

In addition, excessive extracellular matrix (ECM) creates barriers for the therapeutics to travel toward the core of the tumor. Therefore, the classical cancer therapeutics can only impact on reducing the tumor size for a limited duration and once the therapeutic are discontinued tumor recurrence occurs.

Heretofore, the requirements of treating solid tumors while avoiding side effects and other undesirable consequences have not been fully met. In view of the foregoing, there is a need in the art for a solution that simultaneously solves all of these problems.

### SUMMARY

Existing tumor treatment is a mutually exclusive choice between laser therapy or chemotherapy. Laser therapy has a significant limitation in that it can penetrate only as deep as the superficial surface. Chemotherapy has a significant limitation for solid tumor treatment in that the chemotherapeutic drugs cannot penetrate to the deeper layers or regions of the tumor. Moreover, the intra-tumoral pressure pushes back against the drugs. Embodiments of this disclosure can overcome these existing limitations and accelerate solid tumor treatment outcomes in clinical context.

An overall goal of embodiments of the present disclosure is to provide a hybrid laser and chemotherapeutic nanoparticle including a nanosheet such as boron nitride and a BCL-2 inhibitor such as navitoclax. This particular embodiment was further investigated for treating solid tumors and actual laboratory results presented herein. In general, solid tumor treatment is a profound unmet need with the current anticancer drugs because they cannot reach to the core or deeper layer of a tumor. In addition, because of intramural pressure the current anticancer drugs are repulsed from the tumor thereby further compromising therapeutic outcomes.

Embodiments of this disclosure can include a combination of laser and chemotherapeutic modality that can synergistically treat solid tumor by creating a tunnel through the tumor by dissociating extracellular matrix network. Upon laser ablation (heating), the nanoparticles induce dissociation of the proteins and create a porous tunnel to facilitate entrance of the therapeutic regimens to the deeper layers of the tumor. Therefore, this novel, nonobvious and unique approach can be used to advantageously treat solid tumors of many kinds.

An illustrative embodiment of the present disclosure is a composition of matter for treatment of solid tumors, comprising at least one nanosheet; and at least one BCL-2 inhibitor chemically bonded to the at least one nanosheet.

Another illustrative embodiment of the present disclosure is a method of treating a solid tumor, comprising administering to a mammal in need thereof a compound comprising at least one nanosheet; and at least one BCL-2 inhibitor chemically bonded to the at least one nanosheet; and then heating the compound.

Another illustrative embodiment of the present disclosure is a method of making a compound, comprising providing at least one nanosheet; and bonding chemically at least one BCL-2 inhibitor to the at least one nanosheet.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1 illustrates a schematic presentation of BN/NAVI based photothermal therapy in accordance with an illustrative embodiment.

FIGS. 2A-2E illustrate in vitro photo thermal therapy (PTT) effect at 980 nm laser photothermal conversion efficiency in accordance with an illustrative embodiment.

FIG. 3 illustrates thermogravimetric analysis (TGA) in accordance with an illustrative embodiment.

FIG. 4 illustrates X-ray diffraction (XRD) analysis in accordance with an illustrative embodiment.

FIG. 5 illustrates lactate dehydrogenase assay in accordance with an illustrative embodiment.

FIGS. 6A-6B illustrate growth inhibition in 3D spheroids in accordance with an illustrative embodiment.

FIGS. 7A-7B illustrate hydroxyproline and Western blot in tumor spheroids in accordance with an illustrative embodiment.

FIG. 8 illustrates in vivo photo thermal therapy (PTT) effect in accordance with an illustrative embodiment.

FIGS. 9A-9C illustrate tumor morphology, tumor volume and lung metastasis in accordance with an illustrative embodiment.

FIGS. 10A and 10B illustrate hydroxyproline and Western blot assay in tumor tissue in accordance with an illustrative embodiment.

FIG. 11 illustrates a composition of matter in accordance with an illustrative embodiment.

FIG. 12 illustrates a flow diagram of a process for making a compound in accordance with an illustrative embodiment.

FIG. 13 illustrates a flow diagram of a process of treating a tumor in accordance with an illustrative embodiment.

### DETAILED DESCRIPTION

Embodiments of this disclosure include methods and formulations that will ameliorate solid tumors. While not being bound by theory, the methods and formulations induce apoptosis of solid tumor cells.

To facilitate therapeutic penetration into the deeper layers of solid tumors, embodiments of this disclosure include a hybrid therapeutics based on combination of phototherapeutic and chemotherapeutic moeities for synergistic affect and effect. Embodiment of this disclosure can include a 2D boron (BN) nitride nanosheet and a BCL-2 inhibitor, Navitoclax (NAVI). The resulting nanosphere has ability under actinic excitation to breakdown ECS networks to induce pores within the solid tumor. NAVI can be deposited on the surface of BN nanosheet via π-π stacking.

The ability of BN/NAVI to penetrate solid tumor was evaluated on 3D spheroids for the uptake, cytotoxicity, growth inhibition, reactive oxygen species (ROS) detection, penetration and downregulation of proteins in presence, upon laser irradiation. Moreover, irradiated BN/NAVI exhibited significant cytotoxicity, apoptosis and retarded the growth of the spheroids significantly. The BN/NAVI produced ROS enough to kill the cancer cells as confirmed by confocal microscopy. The BN/NAVI penetrated deeper towards the core of the spheroids when irradiated with laser. Westernbloting confirms the BN/NAVI has a therapeutically significant ability to reduce hydroxyproline level and downregulate of BCL-2 protein. Hence, this chemo-phototherapeutic modality (BN/NAVI) is very effective in solid tumor treatment in animals.

The components of the formulation compound can be substituted partially or wholly with other components that provide equivalent functionality. Examples of components that can be substituted include the Table 1 exemplary BCL-2 inhibitors.

TABLE 1
List of BCL-2 inhibitors:

| Inhibitors Name | CAS |
|---|---|
| Navitoclax | 923564-51-6 |
| S55746 hydrochloride | |
| Sabutoclax | |
| Compound Nap-1 | |
| Lisaftoclax | |
| BH3I-1 | |
| Obatoclax Mesylate | 803712-79-0 |
| AZD4320 | |
| Venetoclax | 1257044-40-8 |
| ABT-737 | 852808-04-9 |
| A-1210477 | |
| TW-37 | 877877-35-5 |
| BM-1074 | 1391108-10-3 |
| Gambogic Acid | 2752-65-0 |

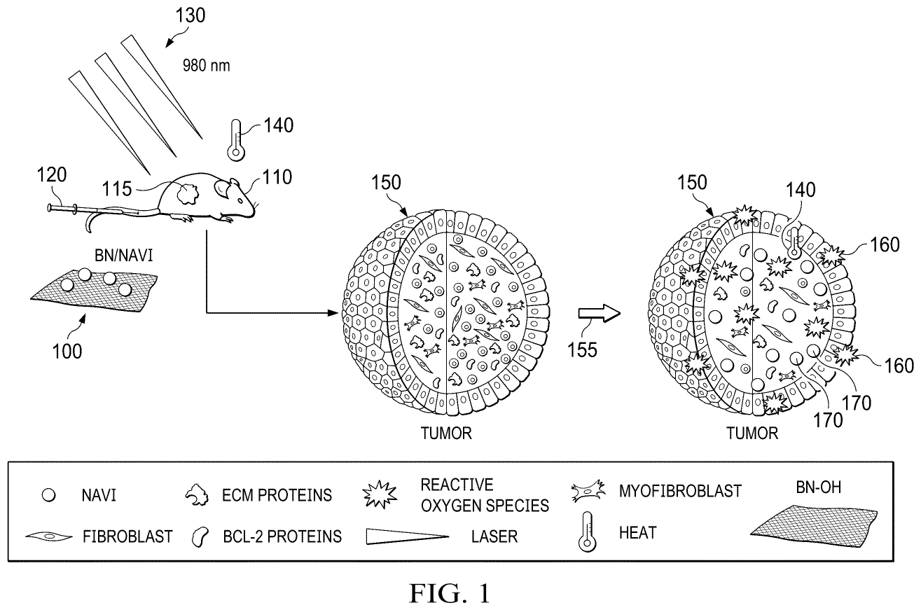

FIG. 1 shows a BN/NAVI based photothermal therapy. A BN/NAVI compound 100 is administered to a mammal 110 having a tumor region 115 by injection 120. A near infrared (NIR) source (not shown) provides 980 nm radiation 130 increasing a temperature 140 of at least a portion of tumor region 115.

Still referring to FIG. 1, individual tumor 150 includes ECM proteins, myofibroblasts, fibroblasts and BCL-2 proteins. After administration of the BN/NAVI compound, exposure to NIR radiation and an elapse of time 155, the tumor has an increased temperature 140 and reactive oxygen species 160 that facilitate penetration of NAVI 170 into the interior of the tumor.

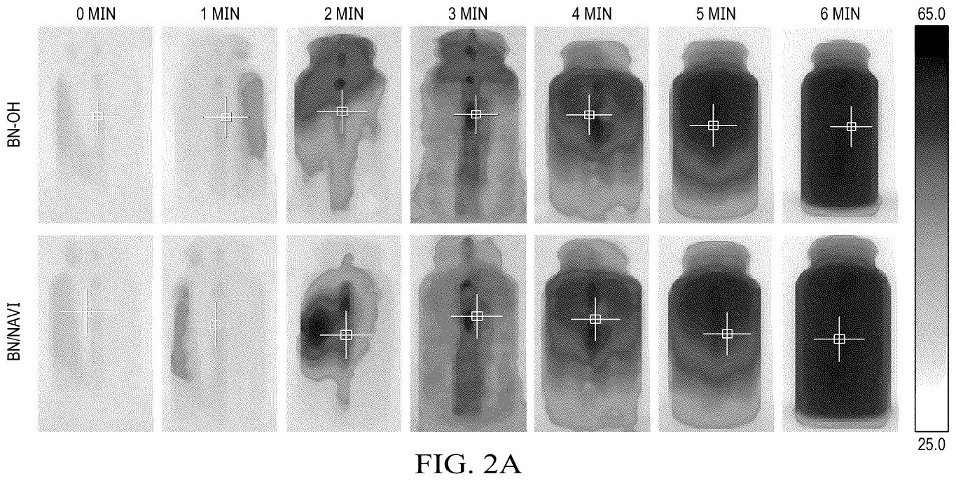

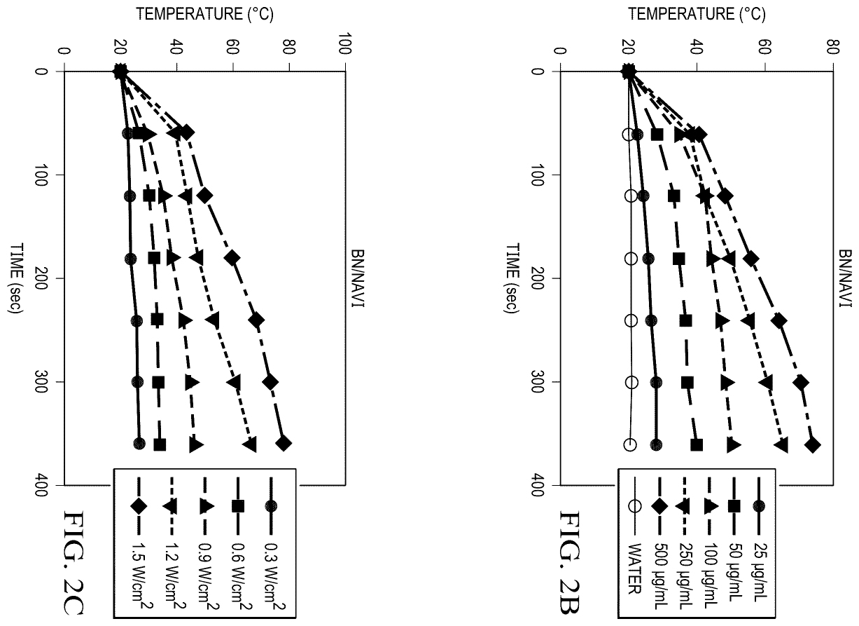

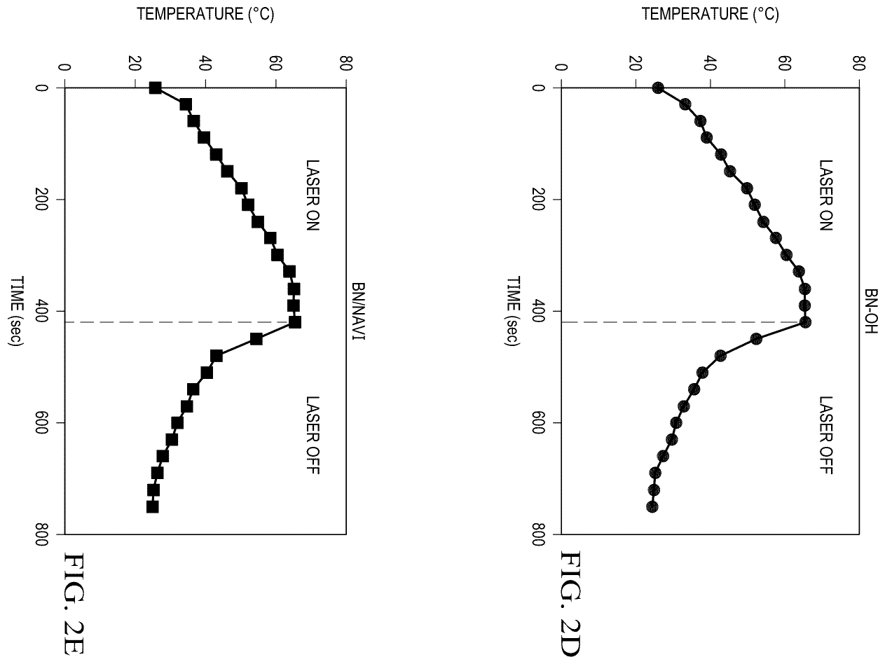

FIGS. 2A-2E show in vitro photo thermal therapy effect. FIG. 2A shows temperature profiles of the solutions of BN—OH and BN/NAVI measured by infrared thermal imaging camera, at one minute intervals as a function of 6 min continuous laser irradiation. FIG. 2B shows temperature profiles of the solutions of BN/NAVI at different concentrations. It can be appreciated that higher concentration provides a higher increase in temperature. FIG. 2C shows temperature profiles of the solutions of BN/NAVI at different laser power density. It can be appreciated that higher power density provides a higher increase in temperature. FIG. 2D shows photothermal stability of BN—OH shown by laser on-off photothermal cycles. FIG. 2E shows photothermal stability of BN-NAVI shown by laser on-off photothermal cycles. It can be appreciated that FIGS. 2D-2E show a relatively linear ramp up in temperature during laser on, followed by an exponential decay in temperature commencing with laser off.

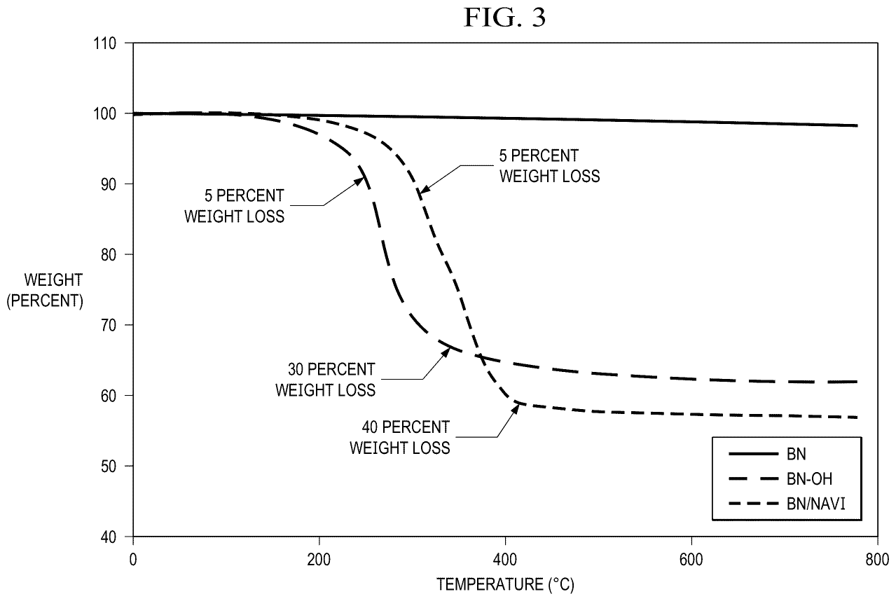

FIG. 3 shows TGA (thermo gravimetric analysis) spectrum of BN, BN—OH, and BN/NAVI. BN is stable. It can be appreciated that BN—OH and BN-NAVI are both initially stable, but that BN—OH starts to lose weight sooner and to a lesser extent than BN-NAVI.

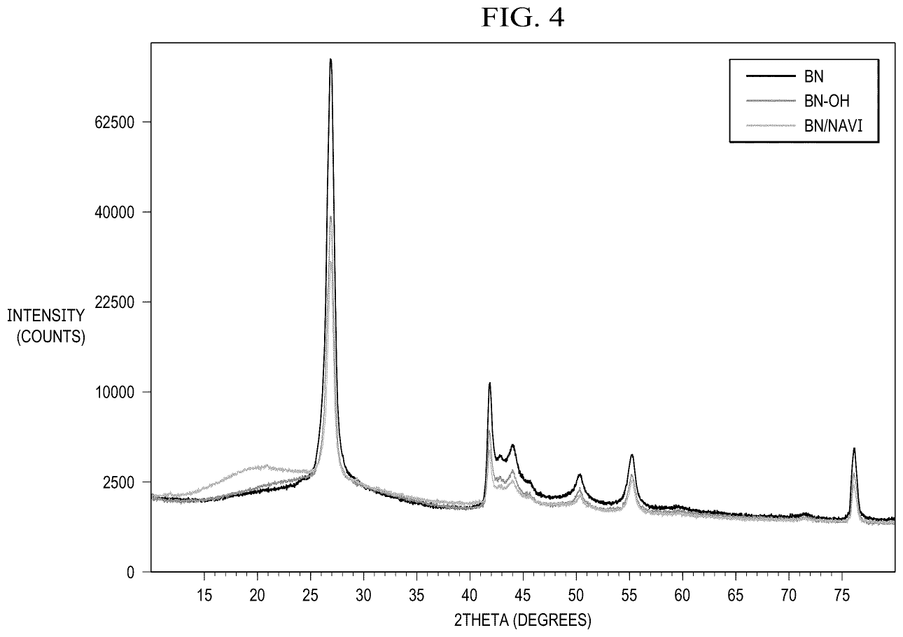

FIG. 4 shows X-ray diffraction (XRD) patterns for BN, BN—OH and BN/NAVI nanoparticles. FIG. 4 provides a definitive description of the BN/NAVI nanoparticles.

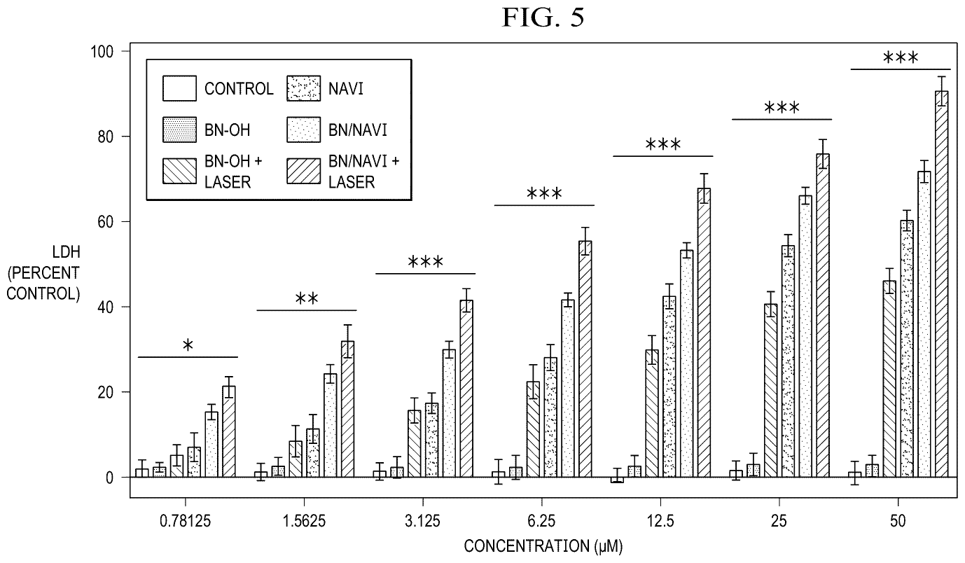

FIG. 5 shows lactate dehydrogenase assay (LDH) release in 3D spheroids treated with BN—OH, NAVI, BN/NAVI, irradiated BN—OH, and BN/NAVI (at NAVI concentrations at 0-50 μM) for 24 h (mean±SD; n=3). The one-way ANOVA (analysis of variance) was used to assess the significance of difference. The symbols *, ** and *** indicate p<0.05, p<0.01 and p<0.001, respectively.

### Annexin-V Assay in 3D Spheroids

Table 2 represents the flow-cytometry of solid tumors treated with various modalities including positive controls (DMSO and H2O2) compared with boron nitride/navitoclax formulations with and without laser excitation. Table 1 demonstrates that almost 90% of cells exhibit apoptosis for the BN/NAVI w/laser group compared to 70% for the same group without laser. This shows clear and solid evidence of the impact of laser excitation on killing cells. The BN formulations without navitoclax also show apoptosis where with laser shows almost 50% apoptosis as opposed to without laser shows only 13% apoptosis.

TABLE 2
Flow-cytometry

| | | | | Free | BN- | BN-OH | BN/ | BN/NAVI |
|---|---|---|---|---|---|---|---|---|
| | Untreated | DMSO | H2O2 | NAVI | OH | w/laser | NAVI | w/laser |
| Live | 99% | 85% | 1% | 58% | 87% | 52% | 30% | 11% |
| Early | 0.06% | 9.51% | 58.37% | 32.04% | 9.22% | 35.62% | 7.5% | 1.51% |
| apoptosis | | | | | | | | |
| Late | 0.02% | 5.23% | 40.12% | 9.64% | 3.34% | 12.63% | 63.3% | 81.08% |
| apoptosis | | | | | | | | |

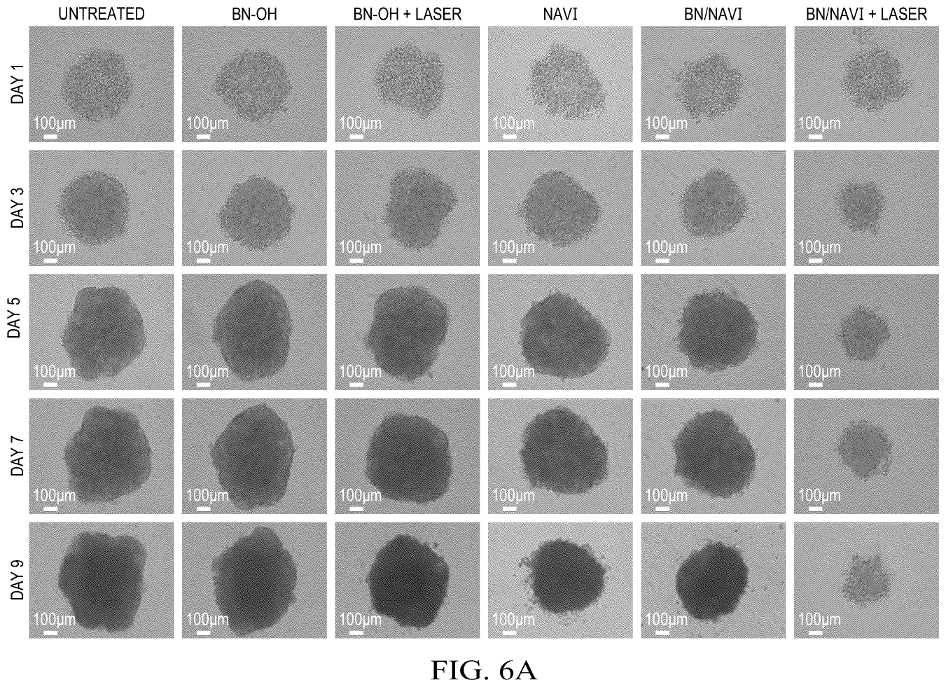

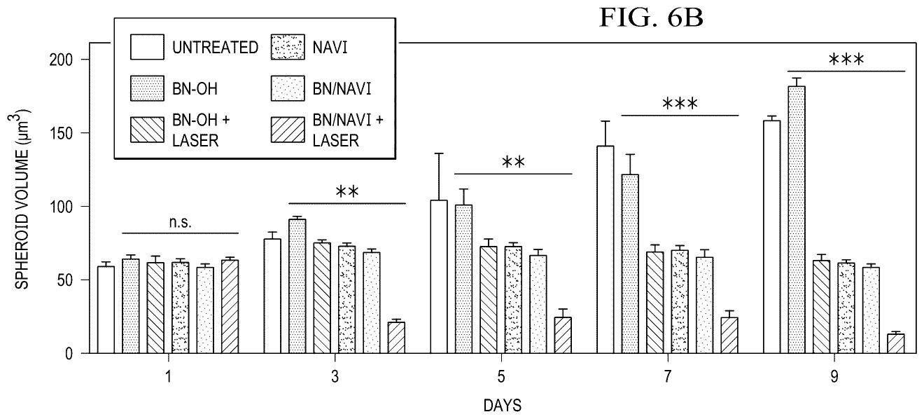

FIGS. 6A-6B show growth inhibition study in spheroids. FIG. 6A shows bright field microscopic images of MDA spheroids untreated and after treatment with BN—OH, irradiated BN—OH, NAVI, BN/NAVI, and BN/NAVI irradiated (at Navi concentration 20 μM) captured at day 1, 3, 5, 7 and 9 at 10× magnification. In the lower left corner of each image is a scale bar of 100 μm. FIG. 6B presents bar graphs showing 3D spheroid growth inhibition (the data presents mean±SD; n=3). One way ANOVA was used to evaluate the statistical significance. n.s., ** and *** indicate p>0.05, p<0.05 and p<0.01 respectively.

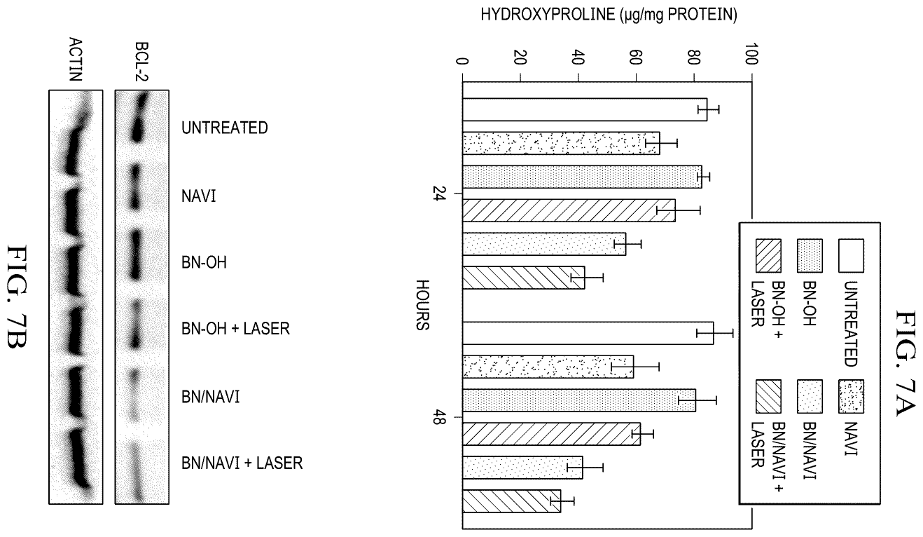

FIG. 7A shows levels of hydroxyproline in 3D spheroids untreated and treated with NAVI, BN—OH, BN/NAVI, irradiated BN—OH, and irradiated BN/NAVI (at NAVI concentration 20 μM) at 24 hours and 48 hours. The data represents mean±SD where n=3. FIG. 7B shows Western blot assay showing BCL-2 expression after treatments. These results show embodiments of this disclosure including irradiated BN/NAVI provide unexpectedly advantageous levels of BCL-2 inhibition.

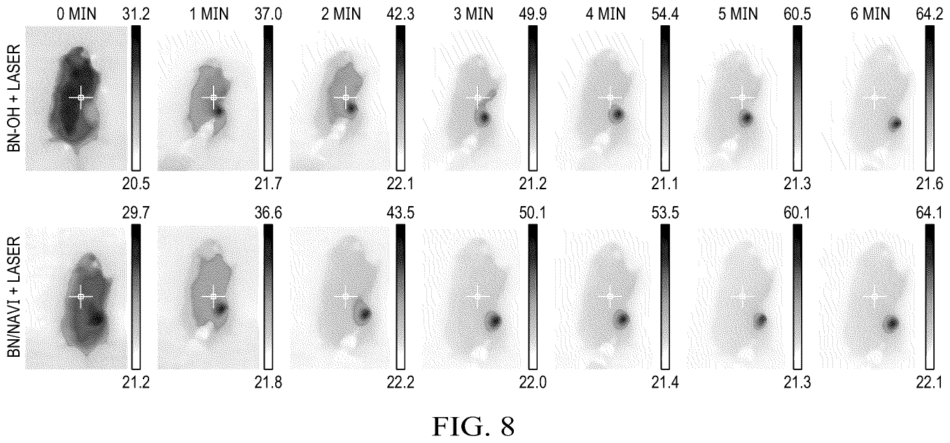

FIG. 8 shows near infrared (NIR) laser irradiated, infrared thermographic maps at from 0 min to 6 min of mice intravenous injected with BN—OH (upper row) and BN/NAVI (lower row).

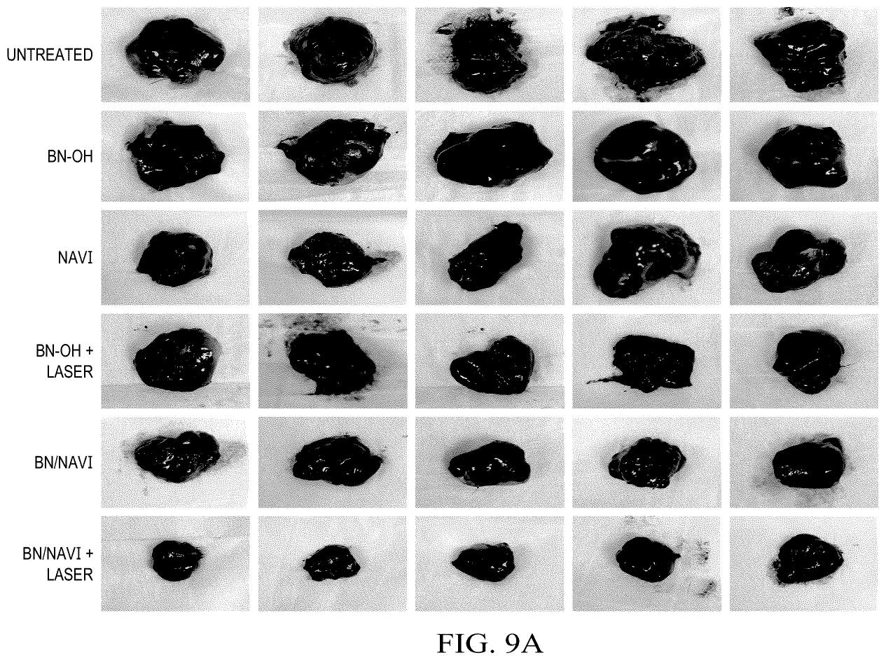

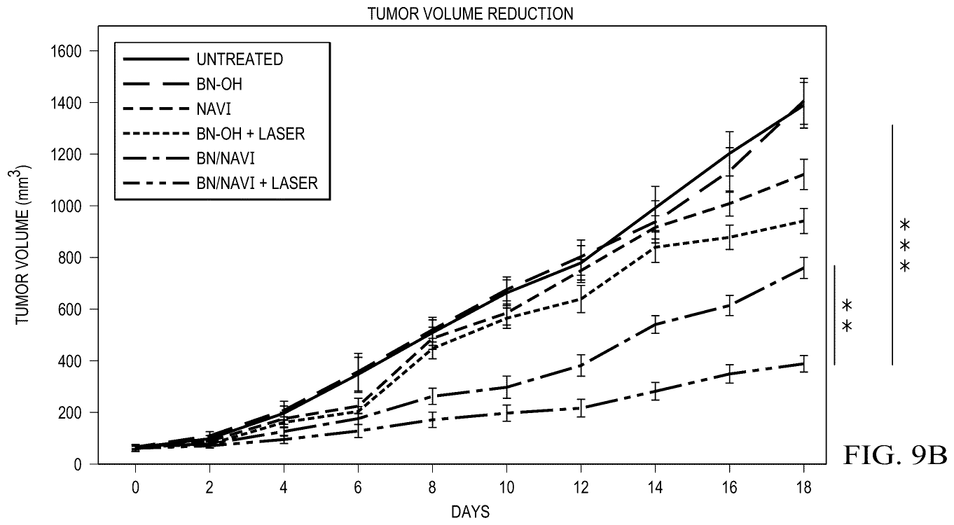

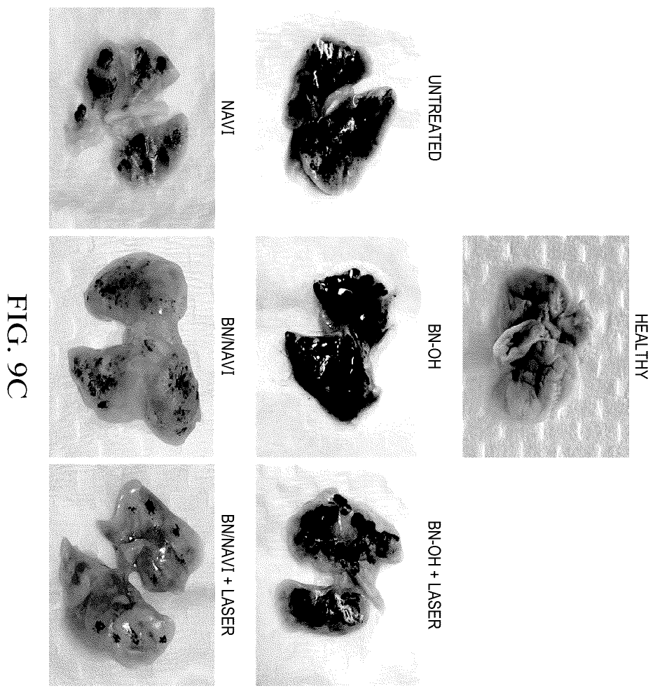

FIGS. 9A-9C show assessment of therapeutic efficacy of untreated, BN—OH, NAVI, BN—OH with laser, BN/NAVI, and irradiated BN/NAVI tumor-bearing C57BL/6 mice. FIG. 9A shows representative appearances of tumors isolated from mice post-treatment. FIG. 9B shows a graphical representation of tumor volume vs. days during treatment. FIG. 9C shows visualization of lungs of mice healthy, untreated and after the treatment with BN—OH, BN—OH with laser, NAVI, BN/NAVI and irradiated BN/NAVI to check the development of metastatic nodules of B16F10 cells. The one-way ANOVA was used to assess the significance of difference. ** and *** indicate p<0.01, p<0.001 respectively.

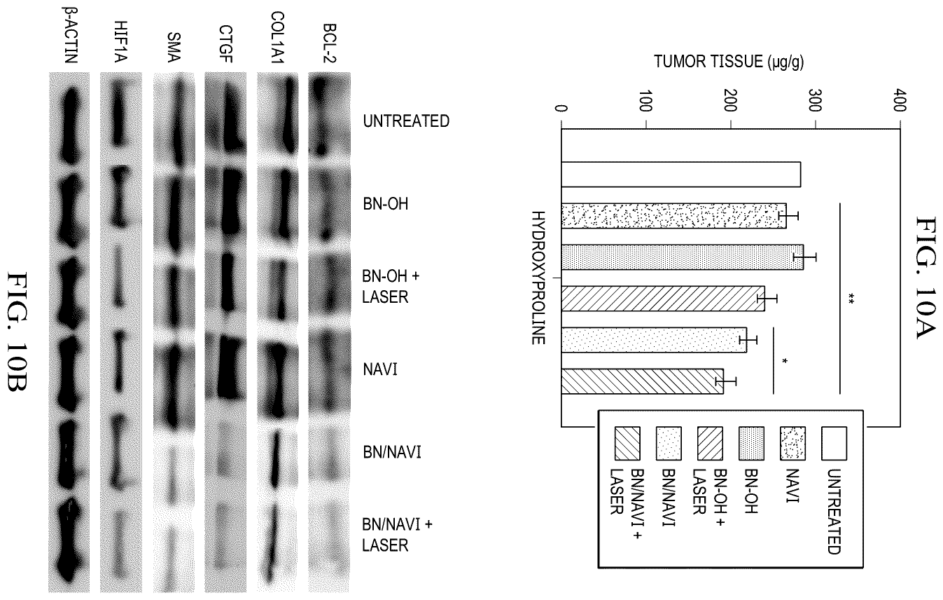

FIG. 10A shows hydroxyproline assay in tumor tissue for untreated, NAVI, BN—OH, irradiated BN—OH, BN/NAVI and irradiated BN/NAVI. FIG. 10B shows Western blot assay in tumor tissue showing expression of various ECM proteins e.g. BCL-2, Collagen1A1, CTGF, SMA, HIF1A and β-ACTIN. The one-way ANOVA was used to assess the significance of difference. * and *** indicate p<0.05, p<0.001 respectively. These results show embodiments including irradiated BN/NAVI provide unexpectedly advantageous levels of BCL-2 inhibition.

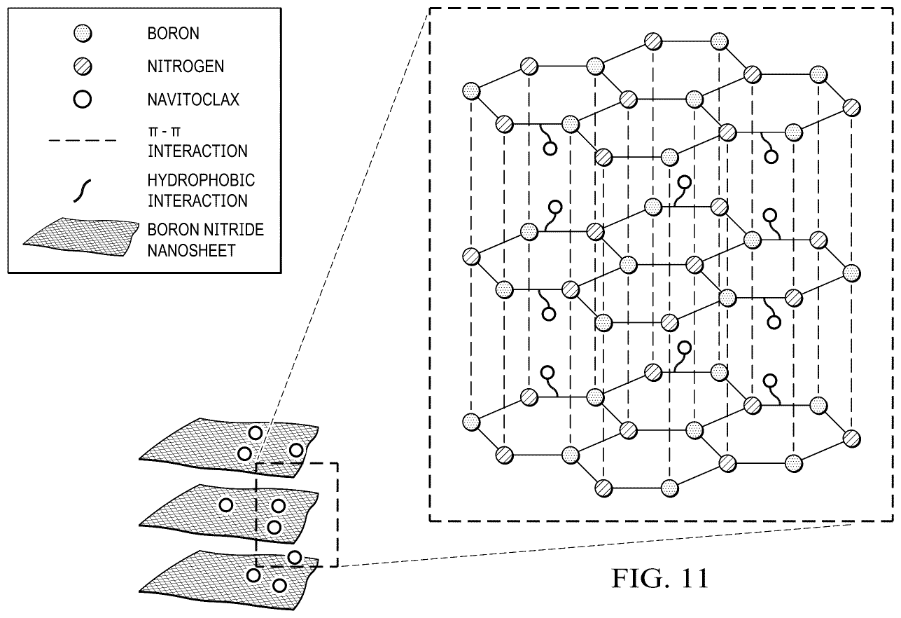

FIG. 11 shows a composition of matter including a nanoparticle including nanosheets 1110 and BCL-2 inhibitors 1120. In this exemplary embodiment, the nanosheets are boron nitride (BN) and the BCL-inhibitors are Navitoclax (NAVI). Without being bound by theory, the dashed lines represent π-π stacking interactions.

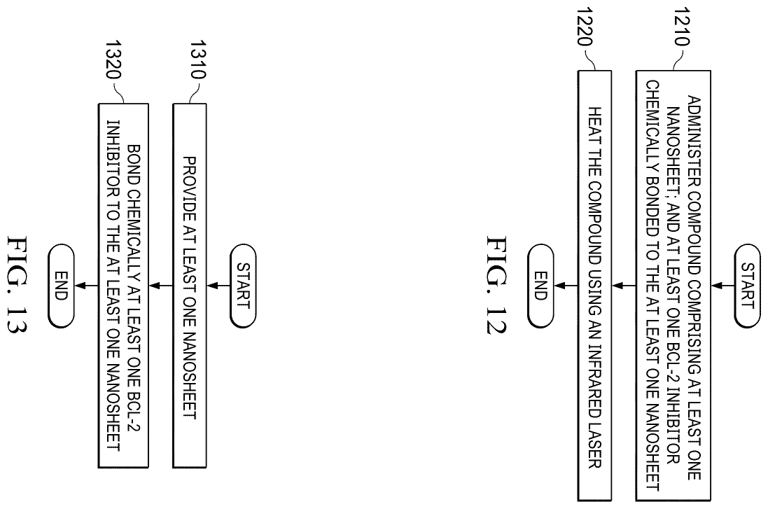

FIG. 12 shows a process for treating a solid tumor. The exemplary process includes at least the following two steps. First, administer to a mammal in need thereof a compound including at least one nanosheet; and at least one BCL-2 inhibitor chemically bonded to the at least one nanosheet 1210. Second, then heat the compound 1220. In general, heating includes directing actinic energy toward the compound where the at least one BCL-2 inhibitor comprises at least one BCL-2 inhibitor selected from the group consisting of Navitoclax, S55746 hydrochloride, Sabutoclax, Compound Nap-1, Lisaftoclas, BH3I-1, Obatoclax Mesylate, AZD4320, Venetoclax, ABT-737, A-1210477, TW-37, BM-1074 or Gambogic Acid. More specifically, heating includes directing laser energy toward the compound where the at least one BCL-2 inhibitor comprises at least one BCL-2 inhibitor selected from the group consisting of Navitoclax, S55746 hydrochloride, Sabutoclax, Compound Nap-1, Lisaftoclas, BH3I-1, Obatoclax Mesylate, AZD4320, Venetoclax, ABT-737 or A-1210477. Heating can include directing infra-red laser energy toward the compound where the at least one BCL-2 inhibitor comprises Navitoclax. Heating can include directing infra-red laser energy toward the compound and the at least one nanosheet comprises boron nitride. Heating can include directing infra-red laser energy toward the compound where the at least one nanosheet comprises a plurality of boron nitride nanosheets coupled to one another by π-π stacking interaction. Heating can include directing infra-red laser energy toward the compound where chemically bonded comprises hydrophobic interaction.

FIG. 13 shows a process of making a compound. This exemplary process includes at least the following two steps. First, provide at least one nanosheet 1310. Second, bond chemically at least one BCL-2 inhibitor to the at least one nanosheet 1320. The process can also include coupling the at least one nanosheet to at least another nanosheet using π-π stacking interaction. The bonding chemically can include hydrophobic interaction. The process can also include locating the compound within a nanosphere. The nanosphere can function as a vehicle for deploying the compound. The process can also include dispersing the nanosphere in a suspension. The process can also include administering the suspension to a mammal in need thereof.

### EXAMPLES

Specific exemplary embodiments will now be further described by the following, nonlimiting examples which will serve to illustrate in some detail various features. The following examples are included to facilitate an understanding of ways in which embodiments of the present disclosure may be practiced. However, it should be appreciated that many changes can be made in the exemplary embodiments which are disclosed while still obtaining like or similar result without departing from the scope of embodiments of the present disclosure. Accordingly, the examples should not be construed as limiting the scope of the present disclosure.

### Example 1

### Method of Preparation:

The hydroxyl functionalization over the BN was performed according to a previously published report. Briefly, the BN was first irradiated in the oven for 2 min and then a potent oxidizing agent hydrogen peroxide was added to the water. A small amount (1% wt) of sulfuric acid was added to this mixture and it was kept for bath sonication overnight to ionize the BN and dissociate the bond between B—N. Next day, the mixture was sonicated using an ultrasonicator for 10 min and this mixture was refluxed overnight at 95° C. to further hydrolyze, which facilitates the further dissociation and conversion to BN—OH from BN. The BN—OH in the mixture was separated from BN using the organic/aqueous solvent extraction process, in which the aqueous phase contains BN—OH and the chloroform phase contains BN, and it was freeze-dried.

In the next step, the NAVI was adsorbed over the surface of exfoliated BN—OH particles by sonication-assisted adsorption. Briefly, the NAVI was dispersed in 1-5 mL of HEPES buffer and kept for incubation with gentle shaking for 24 hr. Next day, the chloroform was added to the mixture to separate the un-adsorbed NAVI and the aqueous part containing BN/NAVI was subjected to drying under lyophilizer.

### Example 2

### Hypothetical Protocol of Treating Solid Tumor in Humans

This formulation is developed with an aim to treat solid tumors with a combinatorial treatment modality. Embodiments of this disclosure can use hybrid formulation of boron nitride-navitoclax that will expedite and enhance therapeutic outcomes upon administration of light from an external source. We envision that the hybrid nanoparticle will be administered/injected directly to the solid tumors in skin and/or superficial region including breast. Then, a near-infrared light source will be applied on it to provide required energy to the BN to produce adequate heat to dissociate/breakdown the protein network within the extracellular matrix. With dissociation of the ECM, the payload of navitoclax will get passage to penetrate to the deeper tissue/cell and kill the cells from the core of the tumor. The produce heart from the boron nitride will also kill cancer cells via photoablation. Therefore, higher therapeutic outcomes will be due to synergy between the modalities.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition of matter for treatment of solid tumors, comprising:
at least one nanosheet; and
at least one BCL-2 inhibitor chemically bonded to the at least one nanosheet.

2. The composition of claim 1, wherein the at least one BCL-2 inhibitor comprises Navitoclax.

3. The composition of claim 1, wherein the at least one BCL-2 inhibitor comprises at least one BCL-2 inhibitor selected from the group consisting of Navitoclax, S55746 hydrochloride, Sabutoclax, Compound Nap-1, Lisaftoclas, BH3I-1, Obatoclax Mesylate, AZD4320, Venetoclax, ABT-737 or A-1210477.

4. The composition of claim 1, wherein the at least one BCL-2/BCL-xl inhibitor comprises at least one BCL-2 inhibitor selected from the group consisting of Navitoclax, S55746 hydrochloride, Sabutoclax, Compound Nap-1, Lisaftoclas, BH3I-1, Obatoclax Mesylate, AZD4320, Venetoclax, ABT-737, A-1210477, TW-37, BM-1074 or Gambogic Acid.

5. The composition of claim 1, wherein the at least one nanosheet comprises boron nitride.

6. The composition of claim 5, wherein the at least one nanosheet comprises a plurality of boron nitride nanosheets coupled to one another by π-π stacking interaction.

7. The composition of claim 5, wherein chemically bonded comprises hydrophobic interaction.

8. A method of treating a solid tumor, comprising:
administering to a mammal in need thereof a compound comprising:
at least one nanosheet; and
at least one BCL-2 inhibitor chemically bonded to the at least one nanosheet;
and then
heating the compound.

9. The method of claim 8, wherein heating includes directing infra-red laser energy toward the compound and wherein the at least one BCL-2 inhibitor comprises Navitoclax.

10. The method of claim 8, wherein heating includes directing laser energy toward the compound and wherein the at least one BCL-2 inhibitor comprises at least one BCL-2 inhibitor selected from the group consisting of Navitoclax, S55746 hydrochloride, Sabutoclax, Compound Nap-1, Lisaftoclas, BH3I-1, Obatoclax Mesylate, AZD4320, Venetoclax, ABT-737 or A-1210477.

11. The method of claim 8, wherein heating includes directing actinic energy toward the compound and wherein the at least one BCL-2 inhibitor comprises at least one BCL-2 inhibitor selected from the group consisting of Navitoclax, S55746 hydrochloride, Sabutoclax, Compound Nap-1, Lisaftoclas, BH3I-1, Obatoclax Mesylate, AZD4320, Venetoclax, ABT-737, A-1210477, TW-37, BM-1074 or Gambogic Acid.

12. The method of claim 8, wherein heating includes directing infra-red laser energy toward the compound and wherein the at least one nanosheet comprises boron nitride.

13. The method of claim 8, wherein heating includes directing infra-red laser energy toward the compound and the at least one nanosheet comprises a plurality of boron nitride nanosheets coupled to one another by π-π stacking interaction.

14. The method of claim 8, wherein heating includes directing infra-red laser energy toward the compound and wherein chemically bonded comprises hydrophobic interaction.

15. A method of making a compound, comprising:
providing at least one nanosheet; and
bonding chemically at least one BCL-2 inhibitor to the at least one nanosheet.

16. The method of claim 15, further comprising coupling the at least one nanosheet to at least another nanosheet using π-π stacking interaction.

17. The method of claim 15, wherein bonding chemically comprises hydrophobic interaction.

18. The method of claim 15, further comprising locating the compound within a nanosphere.

19. The method of claim 18, further comprising dispersing the nanosphere in a suspension.

20. The method of claim 19, further comprising administering the suspension to a mammal in need thereof.
